(19) United States
(12) Patent Application Publication
(10) Pub. No.: US 2005/0106156 A1
(43) Pub. Date: May 19, 2005

(54) Method for curing mild Multiple Sclerosis, memory impairment and Seasonal Affective Disorder

(76) Inventor: Harvey Lang, Brooklyn, NY (US)

Correspondence Address: EDWARD N. GEWIRTZ;BRONSTEIN, GEWIRTZ & GROSSMAN, LLC, SUITE 4600, 60 EAST 42ND STREET, NEW YORK, NY, 10165, US

(21) Appl. No.: 10/714,042

(22) Filed: Nov. 17, 2003

(51) Int. Cl. A61K 39/00

(52) U.S. Cl. 424/184.1

(57) ABSTRACT

A method for curing mild Multiple Sclerosis, memory impairment and Seasonal Affective Disorder. An Applied Kinesiology technique, used to desensitize a patient to allergies, is used to desensitize a patient to myelin and melatonin. Desensitizing the patient to myelin and melatonin cures mild Multiple Sclerosis, memory impairment and Seasonal Affective Disorder.

### FIELD OF THE INVENTION

This invention relates to an Applied Kinesiology method for curing mild Multiple Sclerosis, memory impairment and Seasonal Affective Disorder.

### BACKGROUND

Seasonal Affective Disorder (“SAD”) is a phenomenon whereby many people become sad and depressed during the winter months. People tend to have elevated levels of melatonin in their bodies during the winter months due to the lesser amount of daylight in the winter months. Melatonin is a chemical that is generated by the body in dark conditions. During the winter months there is less daylight, which leads to the body's production of greater amounts of melatonin, and thus to the body having an elevated melatonin level in the dark, winter months.

The inventor, an applied kinesiologist, has found that people who suffer from SAD have a sensitivity to the elevated melatonin level in their body, which causes them to have an allergic type reaction of depression. SAD, the inventor hypothesizes, is caused by the body's allergic type reaction to the elevated melatonin level, which manifests itself as depression. Prior methods for attempting to treat SAD have recognized that SAD is connected with, inter alia, elevated melatonin levels in the body and have tried to decrease the melatonin levels by exposing SAD patients to artificial light sources to compensate for the lack of sunlight exposure. See, for example, U.S. Pat. No. 5,447,528 by Gerardo.

The inventor herein has invented a method for curing SAD, by desensitizing SAD patients to melatonin using allergy desensitization techniques that are known in the field of applied kinesiology. The inventor has found that his patients who suffered from SAD had improved in their condition after desensitizing those patients to melatonin.

Patients with SAD will show sensitivity to melatonin. This sensitivity may be detected by use of well known Applied Kinesiology methods for testing for the temporary weakening of a patient's muscles. As in known in the field of applied kinesiology, when a patient is exposed to a substance that the patient is sensitive to, which in the case of a patient suffering from SAD is melatonin, the patient's muscles will be temporarily weakened.

The muscle weakness test may involve simply having a patient hold a homeopathic vial containing melatonin in the patient's hand, while the clinician then manually tests the patient's muscle strength. For example, the clinician can test for changes in the patient's ability to hold up the patient's elbow while the clinician tries to push the patient's elbow downward. The clinician can more easily press down the SAD patient's elbow when the SAD patient holds in his/her hand a homeopathic vial containing melatonin.

The inventor further found that all of his patients who suffer from SAD also show an allergic type sensitivity to myelin. The allergic type sensitivity to myelin can be treated by using the same applied kinesiology technique, only with myelin rather than melatonin. The allergic type reaction to myelin causes upper neural lesioning of myelinated nerves, which impairs the patient's memory and causes mild Multiple Sclerosis. Desensitizing the patient to myelin will cure the memory impairment and the mild Multiple Sclerosis.

The inventor's hypothesis is that the SAD patient's allergic type reaction to melatonin results from and stems from the patient's allergic type reaction to myelin. The allergic type reaction to myelin causes upper neural lesioning of myelinated nerves. During the day, when a patient is conscious, there is a conscious avoidance by the patient's body of weak pathways for transmission of information, i.e. an avoidance of damaged myelinated nerves for transmission of information. However at night time, when a patient sleeps and dreams, there is no conscious avoidance of weak pathways for transmission of information over myelinated nerves, and memories are more apt to attempt to travel through the diseased, myelin attacked nerves. At night time there is also an increased presence of melatonin in the body, which functions in the body quantitatively more at night and during sleep. The body thus comes to associate the elevated melatonin level with disfunction, i.e. with the attempted transmission of memories over damaged, myelinated nerves. The body's association of melatonin with disfunction leads the body to want to attack the melatonin, leading the body to develop an allergic type reaction to melatonin develops. The allergic type reaction causes SAD.

As stated above, the effect of the allergic type reaction to myelin in the patient is memory loss and mild Multiple Sclerosis. The mild Multiple Sclerosis can usually be clinically observed by the symptom of the patient having a weak tibialis anticus, often with cog-wheel rigidity illicit on a second muscle test. For example, first the strength of a patient's right tibialis anticus is tested, and will test strong. Immediately afterwards the strength of the left tibialis anticus is tested, and will test weak. Cog-wheel rigidity is when on passive motion of a limb the examiner feels the patient's muscular resistance as a series of jerks, alternating with periods of arrest. After a patient is desensitized to myelin, the patient's cog-wheel rigidity will disappear completely.

### SUMMARY OF THE INVENTION

To cure a patient from mild Multiple Sclerosis, memory impairment and SAD, an allergy desensitization technique is used to desensitize a patient to myelin and melatonin. The allergy desensitization technique is performed as if the patient were allergic to myelin and melatonin. The myelin and melatonin may, though need not, be combined into a single homeopathic vial.

### DESCRIPTION OF THE PREFERRED EMBODIMENT

The curative method for mild Multiple Sclerosis, memory impairment and SAD may be implemented by use of any of a number of allergy desensitization techniques. The preferred allergy desensitization technique known to the inventor for implementing the method for curing SAD is a laser desensitization technique. This technique, in a slightly different form, was originally developed by Dr. Michael Lebowitz. The technique as originally developed by Dr. Lebowitz involved having a patient put an allergen in his mouth or on his philtrum (above the upper lip), muscle testing a previously weak muscle and then applying a laser to four points on the patient's head. The clinician would then test the patient's muscle strength, now showing strength. The clinician would next place a drop of the patient's own blood on the patient's tongue or philtrum, retest the muscle (showing weakness again) and again apply the laser on the four points.

The inventor found as a practical matter that his patients did not like having blood drawn from them, even a small drop. The inventor thus improved upon Dr. Lebowitz' technique by using the patient's saliva placed on the patient's philtrum, rather than the patient's blood. Some of the patient's saliva is collected on a cotton applicator (commonly know as a Q-tip), and the cotton applicator is then placed on the patient's philtrum (the groove just above the center of the upper lip) while the laser is applied to the four points.

The laser should be a red laser of wavelength 635 nm, 650 nm or 675 nm. Other frequencies may also be effective. The patient holds in his/her hand a closed homeopathic vial containing the myelin and melatonin. While the patient holds the vial in his/her hand, (this causes a strong muscle to weaken for a patient with SAD) a laser is applied at four points: just above the two eyebrows, at the anterior fontanel (also called governing vessel 21) and at the posterior fontanel (also called governing vessel 20). The patient's muscle strength is then retested, (showing strong). Now a cotton applicator, dampened by the patient's saliva, is placed on the patient's philtrum, while patient holds the vial (muscle tests weak again) and the laser is again applied to the four point: just above the two eyebrows, at the anterior fontanel (also called governing vessel 21) and at the posterior fontanel (also called governing vessel 20). The muscle is retested, showing strength again.

An alternative allergy desensitization technique that may be used involves applying pressure to a patient's eyes. This technique is fully described in “The Thirty-Second Allergy Cure,” Collected Papers of the Members of the International College of Applied Kinesiology—U.S.A., Volume I, 1988-89, by Harvey Lang. As will be obvious to those skilled in the art, other desensitization techniques may also be used in place of the laser technique, as part of the method for curing SAD.

There is a causative condition of mercury toxicity that has to also be dealt with because it effects the permanence or relative permanence of the cure. There are known drugs, such as Metaplex by Thorne Reasearch, Inc., or Mercury Detox by Tyler (Integrated Therapeutics, Inc.), or even the use of the herb Cilantro, that can be used for correcting mercury toxicity. It is the overload of mercury in the patient's system that generally will cause the allergic type reaction to myelin.

With respect to memory loss, a muscle test can be performed using physical contact with known memory drugs both to diagnose the condition and to show that desensitization to myelin worked to cure the condition. Known memory drugs such as phosphatidylserine capsules, donepezil hydrochloride tablets or aricept phosserine (the “Memory Drugs”) can be used for the muscle test. The clinician elicits a weak muscle from the patient, and observes that the patient's physical contact with the Memory Drugs then cause the weak muscle to strengthen only in patients who suffer from memory impairment. After a patient is desensitized to myelin, the Memory Drugs will no longer cause the weak muscle to strengthen in those same patients, showing that the memory impairment is cured and that the body no longer has need for the Memory Drugs because it is cured.

It is important to point out, however, that not all patients that have a sensitivity or allergy to myelin and melatonin will exhibit the need for Memory Drugs.

1. A method for curing mild Multiple Sclerosis, memory impairment and Seasonal Affective Disorder which comprises:
a clinician desensitizing a patient to myelin and melatonin.

2. The method of claim 1 wherein the clinician desensitizes the patient to myelin and melatonin using an allergy desensitization technique.

3. A method for curing mild Multiple Sclerosis and memory impairment which comprises:
a clinician desensitizing a patient to myelin.

4. The method of claim 3 wherein the clinician desensitizes the patient to myelin using an allergy desensitization technique.

5. A method for curing SAD which comprises:
a clinician desensitizing a patient to melatonin.

6. The method of claim 5 wherein the clinician desensitizes the patient to melatonin using an allergy desensitization technique.
